(19) United States
(12) Patent Application Publication
(10) Pub. No.: US 2004/0171918 A1
(43) Pub. Date: Sep. 2, 2004

(54) Life habit improvement assisting system

(75) Inventors: Hidenori Suzuki, Nagoya-shi (JP); Takashi Nomura, Komaki-shi (JP)

Correspondence Address: OLIFF & BERRIDGE, PLC, P.O. BOX 19928, ALEXANDRIA, VA, 22320, US

(73) Assignee: COLIN MEDICAL TECHNOLOGY CORPORATION, Komaki-shi (JP)

(21) Appl. No.: 10/759,185

(22) Filed: Jan. 20, 2004

(30) Foreign Application Priority Data
Jan. 24, 2003 (JP) 2003-015814

(51) Int. Cl. A61B 5/00

(52) U.S. Cl. 600/300; 128/920; 128/904; 709/213

(57) ABSTRACT

A life habit improvement assisting system, wherein a server apparatus stores first sets of physical information obtained from a first patient, and second sets of physical information obtained from each of second patients, selects, from the second sets of physical information stored for each of the second patients, second sets of physical information obtained from at least one second patient after a prescribed past time point, when a second set of physical information obtained from the one second patient at the prescribed past time point falls within a range determined based on a first set of physical information obtained from the first patient at the prescribed past time point, and when a prescribed target value corresponding to the second set of physical information obtained at the prescribed past time point falls within a range determined based on a prescribed target value corresponding to the first set of physical information obtained at the prescribed past time point, and sends the selected second sets of physical information, and wherein a patient's terminal device receives, from the server apparatus, the selected second sets of physical information, and outputs, for the first patient, first sets of physical information obtained from the first patient after the prescribed past time point, and the received second sets of physical information obtained after the prescribed past time point, for comparison with each other.

### BACKGROUND OF THE INVENTION

1. Field of the Invention

The present invention relates to a life habit improvement assisting system that assists a patient who is instructed by, e.g., a doctor to improve his or her life habit or continue the improved life habit.

2. Related Art Statement

A patient who has the risk of suffering a disease resulting from his or her life habit is instructed by a doctor to improve the life habit. However, it is difficult for the doctor to control the daily life of the patient. Rather, the doctor can only know that the patient keeps, or does not keep, his or her life habit as instructed, only when the patient consults the doctor periodically, e.g., every three months, or occasionally.

Thus, the patient himself or herself needs to control his or her own life habit. There is known an assisting device that assists a patient in keeping his or her life habit, e.g., a physical-information obtaining device that iteratively obtains a plurality of sets of physical information from the patient and displays a time-wise change of the sets of physical information. An example of this physical-information obtaining device is an automatic blood-pressure measuring and recording device disclosed by Japanese Patent Publication No. 2001-190506. The disclosed device includes a printer that records daily changes of blood pressure of a patient, and accordingly the patient can know that his or her blood pressure is improving, is not changing, or worsening. If the blood pressure of the patient is improving as the result of keeping his or her improved life habit, the patient is encouraged to keep further the improved life habit. On the other hand, if the blood pressure is worsening as the result of not improving the life habit, the patient has the feeling of having to improve the left habit. Thus, the disclosed device assists the patient in improving his or her life habit or keeping the improved life habit.

Every patient needs a strong will in keeping improved life habit on his or her own. Therefore, if the patient can only see a time-wise change of his or her physical information, as taught by the above-indicated document, the patient may not be able to maintain his or her will to keep the improved life habit and may even give up the same.

### SUMMARY OF THE INVENTION

It is therefore an object of the present invention to provide a life habit improvement assisting system that assists a patient in improving life habit or keeping the improved life habit.

The above object has been achieved by the present invention. According to a first aspect of the present invention, there is provided a life habit improvement assisting system, comprising a first physical-information obtaining device which iteratively obtains a plurality of first sets of physical information from a first patient; a plurality of second physical-information obtaining devices each of which iteratively obtains a plurality of second sets of physical information from a corresponding one of a plurality of second patients; a server apparatus including (a) a memory device which stores the first sets of physical information obtained from the first patient by the first physical-information obtaining device, and the second sets of physical information obtained from each of the second patients by a corresponding one of the second physical-information obtaining devices, such that the first sets of physical information are associated with the first patient and the second sets of physical information is associated with the each of the second patients, and (b) a first sending means for selecting, from the second sets of physical information stored for the each of the second patients by the memory device, a plurality of second sets of physical information obtained from at least one second patient after a prescribed past time point, when a second set of physical information obtained from the at least one second patient at the prescribed past time point falls within a first range determined based on a first set of physical information-obtained from the first patient at the prescribed past time point, and when a second prescribed target value corresponding to the second set of physical information obtained at the prescribed past time point falls within a second range determined based on a first prescribed target value corresponding to the first set of physical information obtained at the prescribed past time point, and sending the selected second sets of physical information obtained after the prescribed past time point; and a first patient's terminal device including (c) a first receiving means for receiving, from the sending means of the server apparatus, the selected second sets of physical information obtained after the prescribed past time point, and (d) a first output device which outputs, for the first patient, a plurality of first sets of physical information obtained from the first patient after the prescribed past time point and the selected second sets of physical information obtained from the at least one second patient after the prescribed past time point, such that a first time-wise change of the outputted first sets of physical information and a second time-wise change of the outputted second sets of physical information are comparable with each other.

According to the first aspect of the present invention, the first output device outputs the time-wise change of the first sets of physical information obtained from the first patient after the prescribed past time point, and the time-wise change of the second sets of physical information that have been obtained from at least one second patient after the prescribed past time point and meet the conditions that the second set of physical information obtained from the one second patient at the prescribed past time point falls within the first range determined based on the first set of physical information obtained from the first patient at the prescribed past time point, and that the second prescribed target value corresponding to the second set of physical information obtained at the prescribed past time point falls within the second range determined based on the first prescribed target value corresponding to the first set of physical information obtained at the prescribed past time point, so that the two time-wise changes are comparable with each other. Therefore, the first patient can compare the time-wise change of his or her own physical information or index with that of at least one different person (i.e., at least one second patient) who is likewise engaged in improving life habit. Thus, the first patient has the feeling of competition with the different person, and is encouraged by knowing that it is not only him or her who is engaged in improving life habit. Thus, the first patient can easily keep or continue his or her improved life habit.

According to a preferred feature of the first aspect of the present invention, the assisting system further comprises at least one second patient's terminal device corresponding to the at least one second patient, wherein the server apparatus further comprises a second sending means for sending the first sets of physical information obtained from the first patient after the prescribed past time point, and stored by the memory device, to the at least one second patient's terminal device, and wherein the at least one second patient's terminal device comprises (e) a second receiving means for receiving the first sets of physical information obtained after the prescribed past time point, and sent from the second sending means, and (f) a second output device which outputs, for the at least one second patient, the first sets of physical information obtained from the first patient after the prescribed past time point, and received by the second receiving means, and the second sets of physical information obtained from the at least one second patient after the prescribed past time point, and selected by the first sending means, such that the first time-wise change of the outputted first sets of physical information and the second time-wise change of the outputted second sets of physical information are comparable with each other. According to this feature, not only the first patient can compare the time-wise change of his or her own physical information with that of at least one different person (i.e., at least one second patient) who is likewise engaged in improving life habit, but also the different person or second patient can compare the time-wise change of his or her own physical information with that of the first patient who is likewise engaged in improving life habit. Thus, the second patient has the feeling of competition with the first patient, and is encouraged by knowing that it is not only him or her who is engaged in improving life habit. Thus, the second patient is also assisted in keeping or continuing his or her improved life habit.

According to a second aspect of the present invention, there is provided a server apparatus for use in a life habit improvement assisting system, the apparatus comprising a memory device which stores a plurality of first sets of physical information iteratively obtained from a first patient by a first physical-information obtaining device and iteratively sent from the first physical-information obtaining device, and a plurality of second sets of physical information iteratively obtained from each of a plurality of second patients by a corresponding one of a plurality of second physical-information obtaining devices and iteratively sent from the one second physical-information obtaining device; and a sending means for selecting, from the second sets of physical information stored for the each of the second patients by the memory device, a plurality of second sets of physical information obtained from at least one second patient after a prescribed past time point, when a second set of physical information obtained from the at least one second patient at the prescribed past time point falls within a first range determined based on a first set of physical information obtained from the first patient at the prescribed past time point, and when a second prescribed target value corresponding to the second set of physical information obtained at the prescribed past time point falls within a second range determined based on a first prescribed target value corresponding to the first set of physical information obtained at the prescribed past time point, and sending the selected second sets of physical information obtained after the prescribed past time point, and a plurality of first sets of physical information obtained from the first patient after the prescribed past time point, and stored by the memory device, to an output device which outputs, for the first patient, the second sets of physical information obtained after the prescribed past time point, and the first sets of physical information obtained after the prescribed past time point, such that the outputted first sets of physical information and the outputted second sets of physical information are comparable with each other.

According to a preferred feature of the second aspect of the present invention, the sending means selects, from the second sets of physical information stored for the each of the second patients by the memory device, a plurality of second sets of physical information obtained from each of a plurality of second patients after the prescribed past time point, and sends the selected second sets of physical information obtained from the each of the second patients after the prescribed past time point. According to this feature, the first output device outputs the time-wise change of the second sets of physical information that have been obtained from a plurality of second patients after the prescribed past time point, such that the time-wise change of the second sets of physical information can be compared with the time-wise change of the first sets of physical information obtained from the first patient after the prescribed past time point. Thus, the first patient can compare the time-wise change of his or her own physical information with those of a plurality of different persons (i.e., the plurality of second patients) who are likewise engaged in improving life habit. Thus, the first patient has the stronger feeling of competition with those persons, and is more strongly encouraged. Thus, the first patient can more easily continue his or her further improved life habit.

According to a third aspect of the present invention, there is provided an output control program for use in a life habit improvement assisting system, the program comprising a physical-information receiving means for receiving, via a communication line, a plurality of first sets of physical information iteratively obtained from a first patient after a prescribed past time point, and a plurality of second sets of physical information iteratively obtained from at least one second patient after the prescribed past time point, when a second set of physical information obtained from the at least one second patient at the prescribed past time point falls within a first range determined based on a first set of physical information obtained from the first patient at the prescribed past time point, and when a second prescribed target value corresponding to the second set of physical information obtained at the prescribed past time point falls within a second range determined based on a first prescribed target value corresponding to the first set of physical information obtained at the prescribed past time point, and an output control means for operating an output device to output the first and second sets of physical information obtained after the prescribed past time point, and received by the receiving means, such that a first time-wise change of the outputted first sets of physical information and a second time-wise change of the outputted second sets of physical information are comparable with each other.

### BRIEF DESCRIPTION OF THE DRAWINGS

The above and optional objects, features, and advantages of the present invention will be better understood by reading the following detailed description of the preferred embodiments of the invention when considered in conjunction with the accompanying drawings, in which:

FIG. 1 is a view showing a construction of a life habit improvement assisting system to which the present invention is applied;

FIG. 2 is a view showing a construction of a server apparatus of the system shown in FIG. 1;

FIG. 3 is a diagrammatic view for explaining essential control functions of a CPU (central processing unit) of the server apparatus shown in FIG. 2;

FIG. 4 is a view showing a construction of a patient's terminal device of the system shown in FIG. 1;

FIG. 5 is a diagrammatic view for explaining essential control functions of a CPU of the patient's terminal device;

FIG. 6 is a view showing an example of a set of output information that is displayed by a display device under control of an output control means shown in FIG. 6;

FIG. 7 is a flow chart for explaining the essential control functions of the CPU of the server apparatus shown in FIG. 2; and

FIG. 8 is a flow chart for explaining the essential control functions of the CPU of the patient's terminal device shown in FIG. 4.

### DETAILED DESCRIPTION OF PREFERRED EMBODIMENTS

Hereinafter, there will be described a preferred embodiment of the present invention in detail by reference to the drawings.

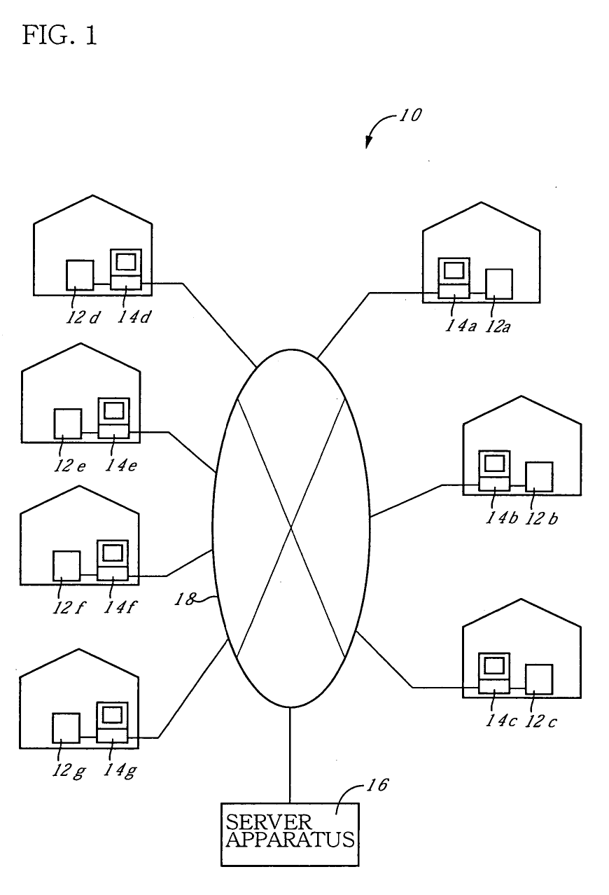

FIG. 1 shows a construction of a life habit improvement assisting system 10 as an embodiment of the present invention. The life habit improvement assisting system 10 shown in FIG. 1 includes a plurality of physical information obtaining devices 12 (12a, 12b, . . . , 12g) that are disposed in, e.g., a plurality of patient's houses, respectively, and obtain a plurality of sets of physical information from a plurality of patients, respectively; a plurality of patient's terminal devices 14 that are connected to the plurality of physical information obtaining devices 12, respectively, and are operated by the plurality of patients, respectively; a server apparatus 16 that is disposed in, e.g., a hospital, or a management company that manages the present system 10; and a communication line 18.

Each of the physical information obtaining devices 12 (12a, 12b, . . . , 12g) obtains, from a corresponding one of the patients, a set of physical information representing a prescribed number of common sort or sorts of physical index or indices that is or are selected in advance from a plurality of sorts of physical indices each of which is influenced by the improvement of patient's life habit, e.g., blood pressure, BP; body weight, W; body fat percentage; body mass index, BMI; and pulse wave propagation velocity, PWV. The prescribed number may be singular or plural. Body mass index BMI is an index of conformation of body, and is defined as a figure obtained by dividing body weight W (kilograms) by square of stature (meters).

An arbitrary one of the physical information obtaining devices 12 may provide a first physical information obtaining device which obtains a set of physical information from a first patient; and the other physical information obtaining devices 12 may provide second physical information obtaining devices which obtain respective sets of physical information from second patients. As far as the present embodiment is concerned, it is assumed that the physical information obtaining device 12a provides the first physical information obtaining device, and the other physical information obtaining devices 12b, . . . , 12g provide the second physical information obtaining devices.

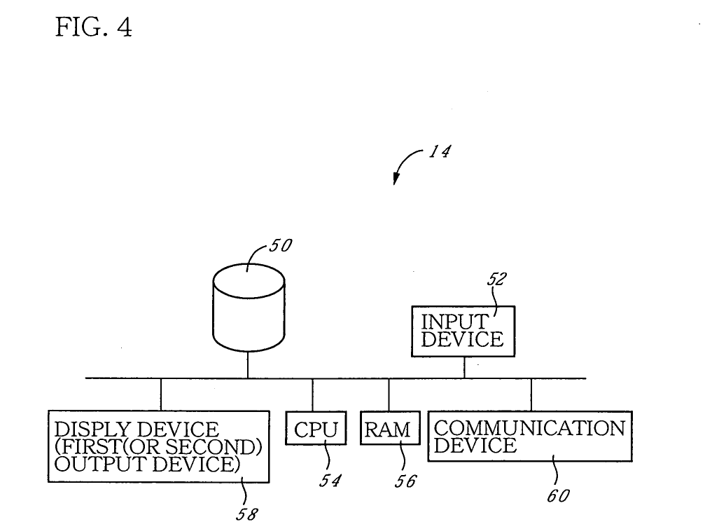

Each of the patient's terminal devices 14 (14a, 14b, . . . , 14g) is provided by a computer having a communication function, and is operated by a corresponding one of the patients. Each of the patient's terminal devices 14 stores each set of physical information supplied from a corresponding one of the physical information obtaining devices 12; sends the stored each set of physical information to the server apparatus 16 via the communication line 18; receives a set of output information sent from the server apparatus 16; and operates a display device 58 thereof (FIG. 4) to display a screen image representing the received set of output information. The patient's terminal devices 14a, 14b, . . . , 14g have the same functions. Since, in the present embodiment, it is assumed that the physical information obtaining device 12a provides the first physical information obtaining device and the other physical information obtaining devices 12b, . . . , 12g provide the second physical information obtaining devices, it is assumed that the patient's terminal device 14a provides a first patient's terminal device connected to the first physical information obtaining device and the other patient's terminal devices 14b, . . . , 14g provide second patient's terminal devices connected to the second physical information obtaining devices, respectively.

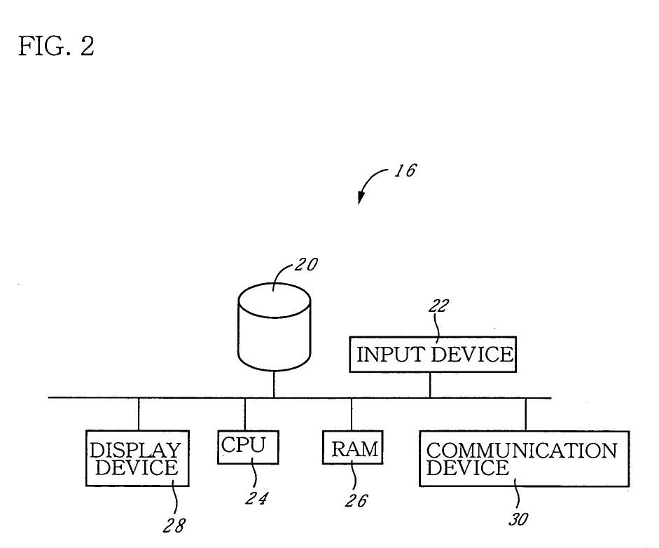

The communication line 18 is provided by a wire or wireless internet or LAN. The server apparatus 16 is provided by a high-speed and high-capacity computer and includes, as shown in FIG. 2, a hard disc 20, an input device 22, a CPU (central processing unit) 24, a RAM (random access memory) 26, a display device 28, and a communication device 30.

The hard disc 20 functions as a memory device, and stores control programs that are used by the CPU 24. In addition, the hard disc 20 stores, for each of the individual patients, one or more sets of physical information sent from a corresponding one of the patient's terminal devices 14, and one or more sets of physical information inputted through the input device 22.

The input device 22 is used by, e.g., a doctor to input one or more target values that is or are set by the doctor for one or more sorts of physical index or indices of a patient who is diagnosed as being in a physical condition in which the patient needs to improve his or her life habit. In addition, the input device 22 is used to input one or more sets of physical information that is or are obtained in the hospital.

The CPU 24 controls the hard disc 20, the display device 28, and the communication device 30, according to the control programs stored in the hard disc 20, while utilizing a temporary-storage function of the RAM 26. The communication device 30 includes a modem, a terminal adaptor, a router, etc. and has the functions of receiving signals sent thereto via the communication line 18 and sending data stored in the hard disc 22 to each of the patient's terminal devices 14 via the communication line 18.

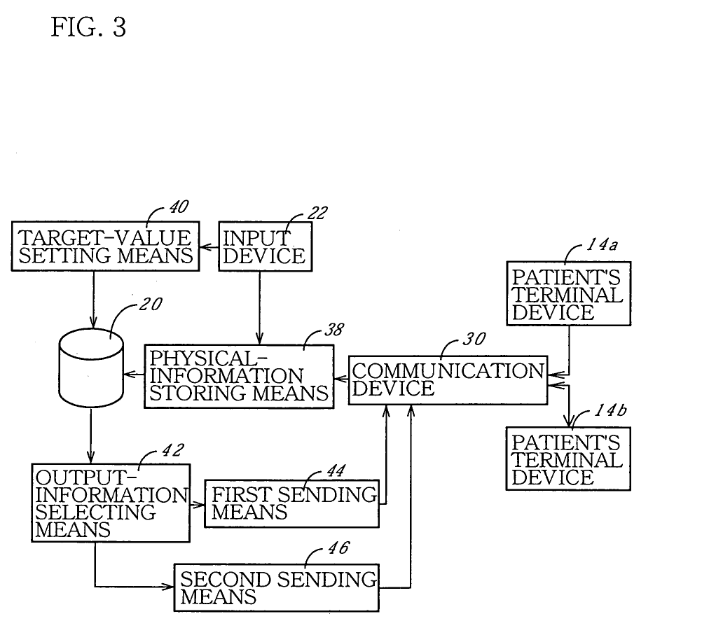

FIG. 3 is a diagrammatic view for explaining essential control functions of the CPU 24 of the server apparatus 16. A physical-information storing device or means 38 stores, in the hard disc 20, and for each of the patients, respective sets of physical information iteratively sent from a corresponding one of the patient's terminal devices 14 and received by the communication device 30, and one or more sets of physical information inputted through the input device 22. A target-value setting device or means 40 sets, for each patient, a target value of each sort of physical index according to a signal inputted through the input device 22, and stores the thus set target value in the hard disc 20. The target value is determined by a doctor based on the patient's age, sexuality, habitus, and other sorts of physical information, and is inputted through the input device 22.

An output-information selecting device or means 42 selects, from first sets of physical information that have been stored, in the hard disc 20, as the sets of physical information obtained from the first patient (i.e., the sets of physical information sent from the first patient's terminal device 14a, and the set of physical information inputted through the input device 22 as the sets of physical information obtained from the first patient), one or more first sets of physical information that has or have been obtained since a prescribed past time point, and additionally selects, from second sets of physical information that have been stored, in the hard disc 20, as the sets of physical information obtained from the second patients (i.e., the sets of physical information sent from the second patient's terminal device 14b, . . . , 14g, and the sets of physical information inputted through the input device 22 as the sets of physical information obtained from the second patients), one or more second sets of physical information that has or have been obtained since the prescribed past time point and simultaneously meets or meet the following condition, and finally outputs, as a set of output information, the thus selected first and second sets of information:

The above-indicated condition is such that the second sets of physical information obtained at the prescribed past time point fall within a physical-information range determined based on the first set of physical information obtained at the prescribed past time point, and simultaneously the respective target values corresponding to those second sets of physical information also fall within a target-value range determined based on the target value corresponding to that first set of physical information. The prescribed past time point may be either a number of months (or a number of years or a number of days) before the present time point, e.g., six months before, or a time point when each patient received the last diagnosis made by the doctor, i.e., the set of physical information inputted through the input device 22 was obtained from the each patient by the doctor. The physical-information range determined based on the first set of physical information is such a range that can be regarded as being substantially equal to that first set of physical information, i.e., a range of ±10% of that first set of physical information. The target-value range determined based on the target value corresponding to that first set of physical information is such a range that can be regarded as being substantially equal to that target value, i.e., a range of ±10% of that target value. Thus, if the hard disc 20 stores a plurality of second sets of physical information that meet the above-indicated condition, the output-information selecting means 42 outputs the set of output information including all those second sets of information.

A first sending device or means 44 sends the first and second sets of physical information as the set of output information selected by the output-information selecting means 42, via the communication device 30, to the first patient's terminal device 14a, so that the first and second sets of physical information are displayed on the display device 58 of the terminal device 14a and are compared with each other by the first patient. A second sending device or means 46 sends the set of output information selected by the output-information selecting means 42, via the communication device 30, to the second patient's terminal device or devices 14 (e.g., the terminal device 14b shown in FIG. 3) that had sent, to the server apparatus 16, the second set or sets of physical information included in the set of output information.

Like the server apparatus 16, each of the patient's terminal device 14 includes, as shown in FIG. 4, a hard disc 50 functioning as a memory device, an input device 52, a CPU 54, a RAM 56, a display device 58, and a communication device 60. When the each patient's terminal device 14 functions as the first patient's terminal device 14, the display device 58 thereof functions as a first output device; and when the each patient's terminal device 14 functions as one of the second patient's terminal devices 14, the display device 58 thereof functions as one of second output devices.

The hard disc 50 stores respective sets of physical information iteratively supplied from the physical-information obtaining device 12, and additionally stores an output control program that is used by the CPU 54 to operate the display device 58 to display the set of output information sent from the server apparatus 16.

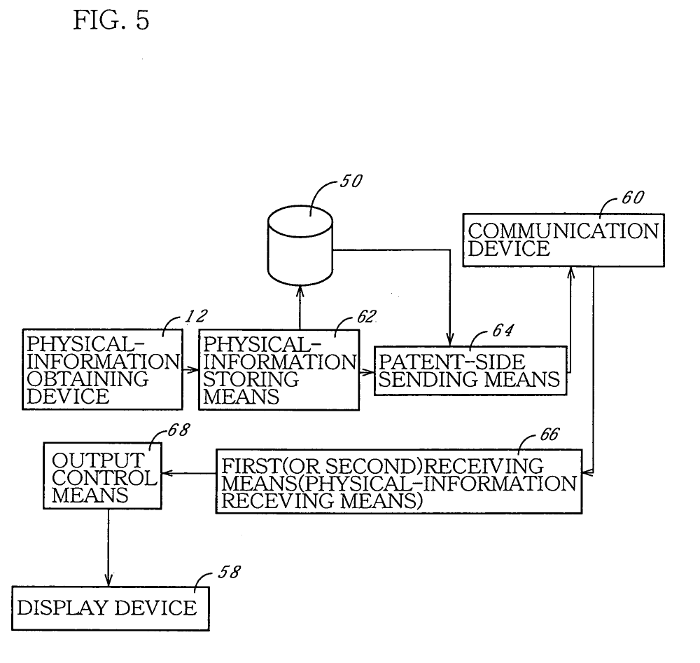

FIG. 5 is a diagrammatic view for explaining essential control functions of the CPU 54 of each of the patient's terminal devices 14. A physical-information storing device or means 62 stores, in the hard disc 50, respective sets of physical information iteratively supplied from the physical-information obtaining device 12. A patient-side sending device or means 64 sends, each time each time the physical-information storing means 62 stores a set of physical information in the hard disc 50, that set of physical information to the server apparatus 16 via the communication device 60.

A receiving device or means 66 and an output control device or means 68 operates according to the output control program stored in the hard disc 50. More specifically described, when each one of the patient's terminal devices 14 functions as the first patient's terminal device 14, the receiving device or means 66 of the each patient's terminal device 14 functions as a first receiving device or means and a physical-information obtaining device or means; and when the each patient's terminal device 14 functions as one of the second patient's terminal devices 14 that receive the set of output information, the receiving device or means 66 of the each patient's terminal device 14 functions as a second receiving device or means and a physical-information obtaining device or means that receives, via the communication device 60, the set of output information sent from the server apparatus 16.

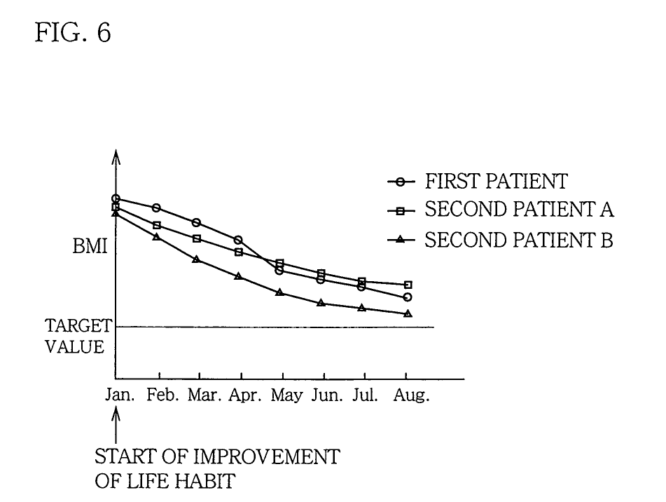

The output control device 68 operates the display device 58 to display the first and second sets of physical information obtained after the prescribed past time point, included in the set of output information received by the receiving means 66, such that a time-wise change of the first sets of physical information and a time-wise change of the second sets of physical information can be compared with each other. FIG. 6 shows an example of the set of output information that is displayed by the display device 58 under control of the output control means 68, more specifically described, a graph including three polygonal lines representing respective time-wise changes of BMI (body mass index) of a first patient and two second patients A, B. However, respective names of the second patients A, B are not shown. Since the second sending means 46, shown in FIG. 3, sends the same set of output information to the respective patient's terminal devices 14 of the second patients A, B, the display device 58 of each of the patient's terminal devices 14 of the second patients A, B shows the same graph as shown in FIG. 6.

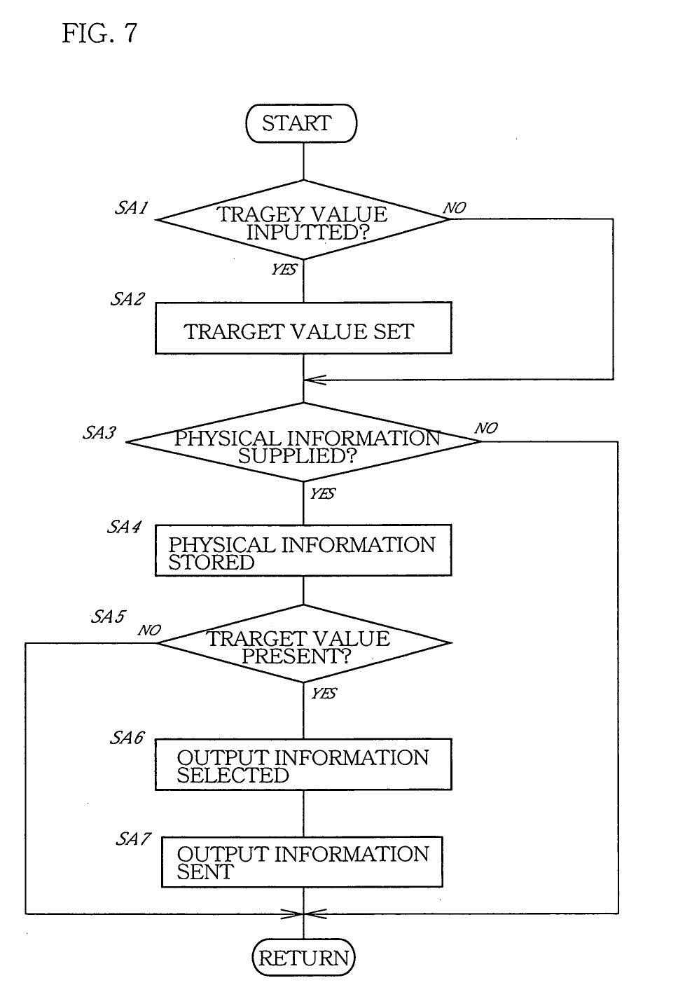

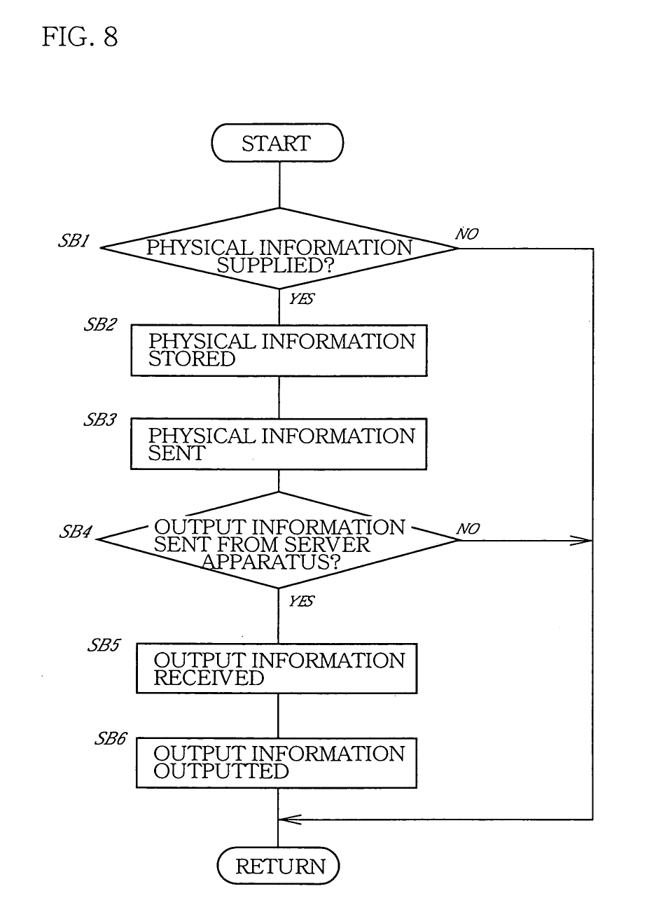

FIG. 7 is a flow chart for explaining the essential control functions of the CPU 24 of the server apparatus 16; and FIG. 8 is a flow chart for explaining the essential control functions of the CPU 54 of each of the patient's terminal devices 14.

In FIG. 7, first, the CPU 24 carries out Steps SA1 and SA2 corresponding to the target-value setting means 40. More specifically described, at Step SA1, the CPU 24 judges whether a signal representing a target value of a physical index, e.g., BMI has been supplied from the input device 22. If a set of physical information obtained from a patient when the doctor makes a diagnosis on the patient is not so bad as to need improvement of his or her life habit, the doctor does not determine a target value of the physical information or index, and accordingly a negative judgment is made. Thus, the control of the CPU 24 proceeds with Step SA3.

On the other hand, if the set of physical information obtained at the time of diagnosis is so bad as to need improvement of life habit of the patient, the doctor instructs the patient to improve his or her life habit, determines a target value of the physical information, and inputs the thus determined target value into the server apparatus 16 through the input device 22. Accordingly, a positive judgment is made at Step SA1. Thus, the control of the CPU 24 proceeds with Step SA2 wherein the CPU 24 sets, for the patient, the target value of the physical information, and stores the thus set target value in the hard disc 20, based on the signal supplied from the input device 22.

When a negative judgment is made at Step SA1, or after Step SA2 is carried out, the CPU 24 carries out Steps SA3 and SA4 corresponding to the physical-information storing means 38. First, at Step SA3, the CPU 24 judges whether a set of physical information has been supplied to the server apparatus 16. If any of the patient's terminal devices 14a, . . . , 14g has supplied a set of physical information to the server apparatus 16 and the communication device 30 of the server apparatus 16 has received the set of physical information, or if the set of physical information obtained at the time of diagnosis has been inputted into the server apparatus 16 through the input device 22, a positive judgment is made at Step SA3, and the control goes to Step SA4. The thus received or inputted set of physical information corresponds to the above-defined first set of physical information obtained from the first patient. In the present embodiment, it is assumed that the first patient is associated with the patient's terminal device 14a, as indicated above. On the other hand, if a negative judgment is made at Step SA3, the control quits this routine.

At Step SA4, the CPU 24 stores, in the hard disc 20, the received or inputted first set of physical information such that each sort of physical information obtained from each patient is identifiable from the other sorts of physical information obtained from the each patient and the other patients and the same sort of physical information obtained from the other patients, i.e., the first set of physical information obtained from the first patient is identifiable from all the second sets of physical information obtained from the second patients and from the other sorts of physical information obtained from the first patient. In the present embodiment, it is assumed that the second patients are associated with the patient's terminal devices 14b, . . . , 14g, respectively, as described above.

Subsequently, at Step SA5, the CPU 24 judges whether a target value has been set for the first set of physical information obtained from the first patient and stored at Step SA4. In the case where the first set of physical information obtained at the time of diagnosis is normal, no target value has been set, and accordingly a negative judgment is made at Step SA5 and the control quits this routine. On the other hand, if a positive judgment is made at Step SA5, the control goes to Step SA6 corresponding to the output-information selecting means 42. At Step SAG, the CPU 24 selects a set of output information from the first and second sets of physical information that have been obtained from the first and second patients and have accumulatively been stored in the hard disc 20 according to this routine. More specifically described, the set of output information includes the first sets of physical information that have been obtained from the first patient after a prescribed past time point, and the second sets of physical information that have been obtained from one or more second patients after the prescribed past time point and meet the above-described condition.

Then, the control goes to Steps SA7 corresponding to the first sending means 44 and the second sending means 46. At Step SA7, the CPU 24 operates the communication device 30 to output the set of output information, selected at Step SAG, to the first patient's terminal device (i.e., the terminal device 14a) and one or more second patient's terminal devices 14 (e.g., the terminal device 14b shown in FIG. 3) associated with the one or more second patients the second sets of physical information obtained from whom are included in the set of output information.

Next, the flow chart shown in FIG. 8 will be explained. First, the CPU 54 carries out Steps SB1 and SB2 corresponding to the physical information storing means 62. At Step SB1, the CPU 54 judges whether a set of physical information has been supplied from the physical-information obtaining device 12. If a negative judgment is made at Step SB1, the control quits this routine. On the other hand, if a positive judgment is made at Step SB1, the control goes to Step SB2 where the CPU 54 stores, in an appropriate memory area of the hard disc 50, the set of physical information supplied from the physical-information obtaining device 12.

Then, the control goes to Step SB3 corresponding to the patient-side sending means 64. At Step SB3, the CPU 54 sends, to the server apparatus 16, the set of physical information stored in the hard disc 50 at Step SB2. Subsequently, the CPU 54 carries out Steps SB4 and SB5 corresponding to the receiving means 66. First, at Step SB4, the CPU 54 judges whether the server apparatus 16 is sending a set of output information to the present patient's terminal device 14. If a negative judgment is made at Step SB4, the control quits this routine. On the other hand, if Step SA7 in FIG. 7 has been carried out and the set of output information has been sent to the present terminal device 14, a positive judgment is made at Step SB4, and the control goes to Step SB5 where the CPU 54 receives the set of output information. Then, at Step SB6 corresponding to the output control means 68, the CPU 54 operates the display device 58 to display the first and second sets of physical information included in the set of output information, e.g., the two diagonal lines, as shown in FIG. 6, representing the first and second sets of physical information, so that the respective time-wise changes of the first and second sets of physical information can be compared with each other by the patient associated with the present terminal device 14.

In the illustrated embodiment, the display device 58 of the first patient's terminal device 14a displays, in the common graph, the two polygonal lines representing the time-wise change of the first sets of physical information obtained from the first patient after the prescribed past time point, and the time-wise change of the second sets of physical information that have been obtained from at least one second patient after the prescribed past time point and meet the condition that the second set of physical information obtained from the one second patient at the prescribed past time point falls within a first range determined based on the first set of physical information obtained from the first patient at the prescribed past time point, and a prescribed target value corresponding to the second set of physical information obtained at the prescribed past time point falls within a second range determined based on a prescribed target value corresponding to the first set of physical information obtained at the prescribed past time point. Therefore, the first patient can compare the time-wise change of his or her own physical information or index with that of different person (i.e., second patient) who is likewise engaged in improving his or her life habit. Thus, the first patient has the feeling of competition with the different person, and is encouraged by knowing that it is not only him or her who is engaged in improving life habit. Thus, the first patient can easily continue his or her improved life habit.

In addition, in the illustrated embodiment, the display device 58 of the first patient's terminal device 14a can display the time-wise change of the second sets of physical information that have been obtained from a plurality of second patients after the prescribed past time point, such that the time-wise change of the second sets of physical information can be compared with the time-wise change of the first sets of physical information obtained from the first patient after the prescribed past time point. Thus, the first patient can compare the time-wise change of his or her own physical information or index with those of a plurality of different persons (i.e., the second patients) who are likewise engaged in improving their life habit. Thus, the first patient has the stronger feeling of competition with those persons, and is more strongly encouraged. Thus, the first patient can more easily continue his or her improved life habit.

Moreover, in the illustrated embodiment, the second sending means 46 (Step SA7) sends the same set of output information as that sent to the first patient's terminal device 14a, to one or more second patient's terminal devices 14 associated with one or more second patients the second sets of physical information obtained from whom are included in the set of output information. Thus, the display device 58 of the one or more second patient's terminal devices 14 displays, in the common graph, the two polygonal lines representing the time-wise change of the first sets of physical information obtained from the first patient after the prescribed past time point, and the time-wise change of the second sets of physical information that have been obtained from the one or more second patients after the prescribed past time point. Thus, not only the first patient can compare the time-wise change of his or her own physical information with that of a different person (i.e., a second patient) who is likewise engaged in improving his or her life habit, but also the different person or second patient can compare the time-wise change of his or her own physical information with that of the first patient who is likewise engaged in improving his or her life habit. Thus, the second patient has the feeling of competition with the first patient, and is encouraged by knowing that it is not only him or her who is engaged in improving life habit. Thus, the second patient is also assisted in continuing his or her improved life habit.

While the present invention has been described in its preferred embodiment by reference to the drawings, it is to be understood that the invention may otherwise be embodied.

For example, in the illustrated embodiment, the output-information selecting means 42 selects the first sets of physical information obtained from the first patient after the prescribed past time point, and the second sets of physical information that have been obtained from the second patient or patients after the prescribed past time point and meet the above-indicated condition; and the first sending means 44 sends, to the first patient's terminal device 14 (i.e., the terminal device 14a), the first and second sets of physical information, selected by the output-information selecting means 42. However, in the illustrated embodiment, the first patient's terminal device 14 (i.e., the terminal device 14a) has, in the hard disc 20, the same first sets of physical information as those selected by the selecting means 42. Therefore, the selecting means 42 may be modified to select the above-indicated second sets of physical information and the first sending means 44 may be modified to send only the thus selected second sets of physical information to the first patient's terminal device 14.

In addition, in the illustrated embodiment, the target value of physical information or index is inputted into the server apparatus 16 through the input device 22. However, the CPU 24 may be modified to determine automatically a target value based on each set of physical information that is stored in the hard disc 20.

In addition, in the illustrated embodiment, the output-information selecting means 42 selects, as the set of output information, all the second sets of information that meet the above-indicated condition. However, it is possible to set an upper limit of the total number of second patients the second sets of physical information obtained from whom are selected by the selecting means 42. Alternatively, it is possible to select the second sets of physical information obtained from only one second patient that are the nearest to the first sets of physical information obtained from the first patient.

In addition, in the illustrated embodiment, the output-information selecting means 42 selects and sends a set of output information, each time the server apparatus 16 stores a set of physical information in the hard disc 20. However, each of the patient's terminal devices 14 may be so modified as to send, to the server apparatus 16, an output-information request signal based on a command inputted through the input device 22, and the server apparatus 16 may be so modified as to select and send a set of output information upon reception of the output-information request signal.

In addition, in the illustrated embodiment, each of the patient's terminal devices 14 displays, in the common graph, the two polygonal lines representing the first and second sets of physical information, so that the respective time-wise changes of the first and second sets of physical information can be compared with each other. However, the polygonal lines may be replaced with other sorts of graphical representation, such as bar graph. Alternatively, the graphical representation may be replaced with a numerical representation consisting of two arrays of numerals representing the respective time-wise changes of the first and second sets of physical information.

The life habit improvement assisting system 10 may be provided with a mailing list that can be used by the first and second patients to encourage each other in improving their life habit.

While the present invention has been described in its preferred embodiments by reference to the drawings, it may be understood that the present invention is by no means limited to the details of the embodiments but may be embodied with various changes and improvements that may occur to a person skilled in the art.

What is claimed is:

1. A life habit improvement assisting system, comprising:
a first physical-information obtaining device which iteratively obtains a plurality of first sets of physical information from a first patient;
a plurality of second physical-information obtaining devices each of which iteratively obtains a plurality of second sets of physical information from a corresponding one of a plurality of second patients;
a server apparatus including (a) a memory device which stores the first sets of physical information obtained from the first patient by the first physical-information obtaining device, and the second sets of physical information obtained from each of the second patients by a corresponding one of the second physical-information obtaining devices, such that the first sets of physical information are associated with the first patient and the second sets of physical information is associated with said each of the second patients, and (b) a first sending means for selecting, from the second sets of physical information stored for said each of the second patients by the memory device, a plurality of second sets of physical information obtained from at least one second patient after a prescribed past time point, when a second set of physical information obtained from said at least one second patient at the prescribed past time point falls within a first range determined based on a first set of physical information obtained from the first patient at the prescribed past time point, and when a second prescribed target value corresponding to the second set of physical information obtained at the prescribed past time point falls within a second range determined based on a first prescribed target value corresponding to the first set of physical information obtained at the prescribed past time point, and sending the selected second sets of physical information obtained after the prescribed past time point; and
a first patient's terminal device including (c) a first receiving means for receiving, from the sending means of the server apparatus, said selected second sets of physical information obtained after the prescribed past time point, and (d) a first output device which outputs, for the first patient, a plurality of first sets of physical information obtained from the first patient after the prescribed past time point and said selected second sets of physical information obtained from said at least one second patient after the prescribed past time point, such that a first time-wise change of the outputted first sets of physical information and a second time-wise change of the outputted second sets of physical information are comparable with each other.

2. The system according to claim 1, further comprising at least one second patient's terminal device corresponding to said at least one second patient, wherein the server apparatus further comprises a second sending means for sending the first sets of physical information obtained from the first patient after the prescribed past time point, and stored by the memory device, to said at least one second patient's terminal device, and wherein said at least one second patient's terminal device comprises (e) a second receiving means for receiving the first sets of physical information obtained after the prescribed past time point, and sent from the second sending means, and (f) a second output device which outputs, for said at least one second patient, the first sets of physical information obtained from the first patient after the prescribed past time point, and received by the second receiving means, and the second sets of physical information obtained from said at least one second patient after the prescribed past time point, and selected by the first sending means, such that the first time-wise change of the outputted first sets of physical information and the second time-wise change of the outputted second sets of physical information are comparable with each other.

3. A server apparatus for use in a life habit improvement assisting system, the apparatus comprising:
a memory device which stores a plurality of first sets of physical information iteratively obtained from a first patient by a first physical-information obtaining device and iteratively sent from the first physical-information obtaining device, and a plurality of second sets of physical information iteratively obtained from each of a plurality of second patients by a corresponding one of a plurality of second physical-information obtaining devices and iteratively sent from said one second physical-information obtaining device; and
a sending means for selecting, from the second sets of physical information stored for said each of the second patients by the memory device, a plurality of second sets of physical information obtained from at least one second patient after a prescribed past time point, when a second set of physical information obtained from said at least one second patient at the prescribed past time point falls within a first range determined based on a first set of physical information obtained from the first patient at the prescribed past time point, and when a second prescribed target value corresponding to the second set of physical information obtained at the prescribed past time point falls within a second range determined based on a first prescribed target value corresponding to the first set of physical information obtained at the prescribed past time point, and sending the selected second sets of physical information obtained after the prescribed past time point, and a plurality of first sets of physical information obtained from the first patient after the prescribed past time point, and stored by the memory device, to an output device which outputs, for the first patient, the second sets of physical information obtained after the prescribed past time point, and the first sets of physical information obtained after the prescribed past time point, such that the outputted first sets of physical information and the outputted second sets of physical information are comparable with each other.

4. The apparatus according to claim 3, wherein the sending means selects, from the second sets of physical information stored for said each of the second patients by the memory device, a plurality of second sets of physical information obtained from each of a plurality of second patients after the prescribed past time point, and sends the selected second sets of physical information obtained from said each of the second patients after the prescribed past time point.

5. An output control program for use in a life habit improvement assisting system, the program comprising:
a physical-information receiving means for receiving, via a communication line, a plurality of first sets of physical information iteratively obtained from a first patient after a prescribed past time point, and a plurality of second sets of physical information iteratively obtained from at least one second patient after the prescribed past time point, when a second set of physical information obtained from said at least one second patient at the prescribed past time point falls within a first range determined based on a first set of physical information obtained from the first patient at the prescribed past time point, and when a second prescribed target value corresponding to the second set of physical information obtained at the prescribed past time point falls within a second range determined based on a first prescribed target value corresponding to the first set of physical information obtained at the prescribed past time point, and
an output control means for operating an output device to output the first and second sets of physical information obtained after the prescribed past time point, and received by the receiving means, such that a first time-wise change of the outputted first sets of physical information and a second time-wise change of the outputted second sets of physical information are comparable with each other.

6. A life habit improvement assisting system, comprising:
a first physical-information obtaining device which iteratively obtains a plurality of first sets of physical information from a first patient;
a plurality of second physical-information obtaining devices each of which iteratively obtains a plurality of second sets of physical information from a corresponding one of a plurality of second patients;
a server apparatus including (a) a memory device which stores the first sets of physical information obtained from the first patient by the first physical-information obtaining device, and the second sets of physical information obtained from each of the second patients by a corresponding one of the second physical-information obtaining devices, such that the first sets of physical information are associated with the first patient and the second sets of physical information is associated with said each of the second patients, and (b) a first sending device which selects, from the second sets of physical information stored for said each of the second patients by the memory device, a plurality of second sets of physical information obtained from at least one second patient after a prescribed past time point, when a second set of physical information obtained from said at least one second patient at the prescribed past time point falls within a first range determined based on a first set of physical information obtained from the first patient at the prescribed past time point, and when a second prescribed target value corresponding to the second set of physical information obtained at the prescribed past time point falls within a second range determined based on a first prescribed target value corresponding to the first set of physical information obtained at the prescribed past time point, and sending the selected second sets of physical information obtained after the prescribed past time point; and
a first patient's terminal device including (c) a first receiving device which receives, from the sending device of the server apparatus, said selected second sets of physical information obtained after the prescribed past time point, and (d) a first output device which outputs, for the first patient, a plurality of first sets of physical information obtained from the first patient after the prescribed past time point and said selected second sets of physical information obtained from said at least one second patient after the prescribed past time point, such that a first time-wise change of the outputted first sets of physical information and a second time-wise change of the outputted second sets of physical information are comparable with each other.

7. The system according to claim 6, further comprising at least one second patient's terminal device corresponding to said at least one second patient, wherein the server apparatus further comprises a second sending device which sends the first sets of physical information obtained from the first patient after the prescribed past time point, and stored by the memory device, to said at least one second patient's terminal device, and wherein said at least one second patient's terminal device comprises (e) a second receiving device which receives the first sets of physical information obtained after the prescribed past time point, and sent from the second sending device, and (f) a second output device which outputs, for said at least one second patient, the first sets of physical information obtained from the first patient after the prescribed past time point, and received by the second receiving device, and the second sets of physical information obtained from said at least one second patient after the prescribed past time point, and selected by the first sending device, such that the first time-wise change of the outputted first sets of physical information and the second time-wise change of the outputted second sets of physical information are comparable with each other.

8. A server apparatus for use in a life habit improvement assisting system, the apparatus comprising:
a memory device which stores a plurality of first sets of physical information iteratively obtained from a first patient by a first physical-information obtaining device and iteratively sent from the first physical-information obtaining device, and a plurality of second sets of physical information iteratively obtained from each of a plurality of second patients by a corresponding one of a plurality of second physical-information obtaining devices and iteratively sent from said one second physical-information obtaining device; and
a sending device which selects, from the second sets of physical information stored for said each of the second patients by the memory device, a plurality of second sets of physical information obtained from at least one second patient after a prescribed past time point, when a second set of physical information obtained from said at least one second patient at the prescribed past time point falls within a first range determined based on a first set of physical information obtained from the first patient at the prescribed past time point, and when a second prescribed target value corresponding to the second set of physical information obtained at the prescribed past time point falls within a second range determined based on a first prescribed target value corresponding to the first set of physical information obtained at the prescribed past time point, and sending the selected second sets of physical information obtained after the prescribed past time point, and a plurality of first sets of physical information obtained from the first patient after the prescribed past time point, and stored by the memory device, to an output device which outputs, for the first patient, the second sets of physical information obtained after the prescribed past time point, and the first sets of physical information obtained after the prescribed past time point, such that the outputted first sets of physical information and the outputted second sets of physical information are comparable with each other.

9. The apparatus according to claim 8, wherein the sending device selects, from the second sets of physical information stored for said each of the second patients by the memory device, a plurality of second sets of physical information obtained from each of a plurality of second patients after the prescribed past time point, and sends the selected second sets of physical information obtained from said each of the second patients after the prescribed past time point.

10. An output control program for use in a life habit improvement assisting system, the program comprising the steps of
receiving, via a communication line, a plurality of first sets of physical information iteratively obtained from a first patient after a prescribed past time point, and a plurality of second sets of physical information iteratively obtained from at least one second patient after the prescribed past time point, when a second set of physical information obtained from said at least one second patient at the prescribed past time point falls within a first range determined based on a first set of physical information obtained from the first patient at the prescribed past time point, and when a second prescribed target value corresponding to the second set of physical information obtained at the prescribed past time point falls within a second range determined based on a first prescribed target value corresponding to the first set of physical information obtained at the prescribed past time point, and
outputting the received first and second sets of physical information obtained after the prescribed past time point, such that a first time-wise change of the outputted first sets of physical information and a second time-wise change of the outputted second sets of physical information are comparable with each other.

11. A recording medium for recording the output control program according to claim 10.
